(19) United States
(12) Patent Application Publication
(10) Pub. No.: US 2015/0233881 A1
(43) Pub. Date: Aug. 20, 2015

(54) APPARATUS FOR DETERMINING PROPERTIES OF A DUST MIXTURE FLOWING THROUGH A CROSS-SECTIONAL AREA OF A COAL DUST LINE

(71) Applicant: SIEMENS AKTIENGESELLSCHAFT, München (DE)

(72) Inventors: Bernhard Meerbeck, Kelkheim (DE); Max Starke, Offenbach A.M. (DE); Jan Weustink, Karlsruhe (DE); Leif Wiebking, Karlsruhe (DE)

(21) Appl. No.: 14/429,792

(22) PCT Filed: Sep. 18, 2013
(86) PCT No.: PCT/EP2013/069349

§ 371 Date: Mar. 20, 2015

(30) Foreign Application Priority Data
Sep. 25, 2012 (DE) 10 2012 217 274.2

(51) Int. Cl. G01N 33/00

(52) U.S. Cl. CPC G01N 33/0027

(57) ABSTRACT

An apparatus for determining properties of a dust mixture flowing through a cross-sectional area of a coal dust line is provided. It has at least one sensor, which has at least one transmitting device for coupling electromagnetic and/or acoustic radiation into the dust mixture and at least one receiving device for generating a measuring signal on the basis of radiation reflected in the dust mixture or radiation transmitted by the dust mixture. Also provided is an evaluation device, which determines the property of the dust mixture on the basis of the measuring signal. The at least one transmitting device and the at least one receiving device or a measuring head are rotatably arranged at least approximately in the middle of the cross-sectional area.

### CROSS-REFERENCE TO RELATED APPLICATIONS

This application claims priority to PCT Application No. PCT/EP2013/069349, having a filing date of Sep. 18, 2013, based on DE 102012217274.2, having a filing date of Sep. 25, 2012, the entire contents of which are hereby incorporated by reference.

### FIELD OF TECHNOLOGY

The following relates to an apparatus for determining properties of a dust mixture flowing through a cross-sectional area of a coal dust line.

### BACKGROUND

In many technical installations, transport by means of a flow of a medium through lines or hoses plays an important part. The medium to be transported is often a multi-phase mixture, which for example consists of a liquid or gaseous carrier medium and a medium additionally to be transported. Examples of a gaseous carrier medium of air with small and extremely small solid and/or liquid particles are flows of dust such as occur in coal-fired power plants. There, for example, the coal dust originating from the coal mills is distributed to multiple burners by way of a multiplicity of coal dust lines.

The more exactly certain properties of a flowing multi-phase mixture are known, such as for example properties of the coal dust in the coal dust lines, the better the underlying process can be influenced, and consequently also optimized. There is therefore always a need for measuring methods that can be widely used and allow the determination of process variables such as mass flow, flow velocity or particle velocity, grain size distribution, moisture and composition of a mixture.

A general problem with the determination of properties, in particular flows of small and extremely small particles, is that of inhomogeneities and uneven distributions both in the direction of flow and in the cross section of the flow. For instance, the distribution of the amounts of coal dust in the coal dust lines, usually formed as pipelines or channels, is influenced by streaming, which cannot be recorded with sufficient resolution by individual measurements.

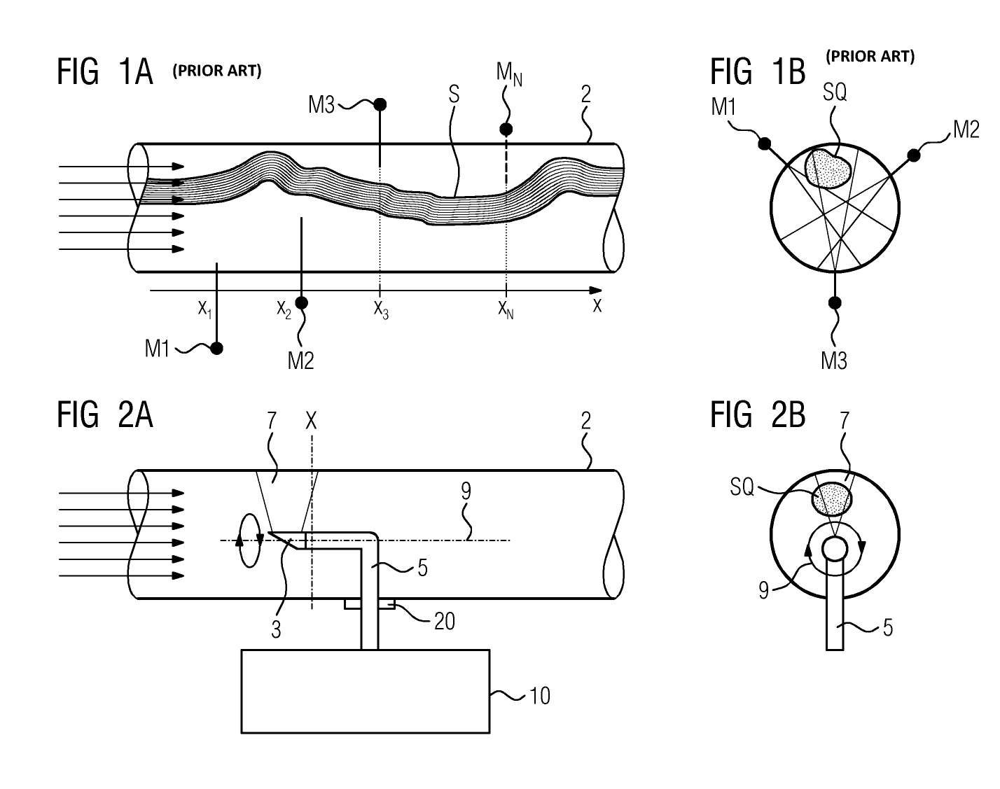

To illustrate the streaming within a pipe, the side view of a straight section of pipe 2 is shown schematically in FIG. 1A. The arrows on the left side indicate the direction of flow of the mixture. Within the section of pipe 2, a streamer S of coal dust is indicated. A typical measuring arrangement consists of a multiplicity of measuring sensors that are arranged to the sides of the section of pipe. At the points x1, x2, x3 and xN, measuring sensors M1, M2, M3 and MN protrude into the interior of the pipe, it being possible for the sensors to be arranged from the outside or else inside the section of pipe, in order on the basis of a specific measuring principle to provide statements for example concerning the burden of the two-phase flow in the form of streamers. In FIG. 1B, the section of pipe 2 is represented in a ghosted view. Three sensors are schematically represented in a 120-degree spatially offset alignment at the points x1 to x3 of the section of pipe. In this representation, it is clear that, due to the fixed arrangement of the measuring sensors in the longitudinal direction of the pipe and on the assumption that a conical radiation is emitted from each sensor, the signal detection is not sufficient in certain regions. For instance, the measuring sensor M2 does not produce any signal, since the streamer cross section denoted by SQ does not lie within the shaded measuring cone of M2. In spite of a large number of sensors, the streaming is only detected by a small proportion, and not by all the sensors. A better resolution, and consequently improved results, can only be achieved in the case of this arrangement by means of additional sensors. A measuring device that is constructed according to the principle shown in FIG. 1A is disclosed in European Patent Specification EP 1 459 055.

Disadvantages of such measuring methods are that they usually measure from the outside, from fixed measuring positions, into or through the measuring volume and that the sensors only have a restricted measuring range, both as far as the depth of penetration into the measuring volume is concerned and as far as the viewing angle is concerned (cf. FIG. 1B). As a result, the spatial resolution, and consequently the accuracy, are limited. The more items of information of a flowing medium are to be recorded, the greater the complexity of the instrumentation, which in turn is accompanied by increased costs.

### SUMMARY

An aspect relates to an apparatus that overcomes the above-mentioned disadvantages. In particular, it is intended to provide a simple setup for the quantitative and spatial recording of inhomogeneities transversely to the direction of flow of a medium, in particular a multi-phase mixture.

Embodiments of the apparatus for determining properties of a medium flowing through a cross-sectional area has at least one sensor, which comprises at least one transmitting device for coupling electromagnetic and/or acoustic radiation into the medium and at least one receiving device for generating a measuring signal on the basis of radiation reflected in the medium or radiation transmitted by the medium. Also provided is an evaluation device, which determines the property of the medium on the basis of the measuring signal. According to embodiments of the invention, the at least one transmitting device and the at least one receiving device are designed for coupling the radiation into and out of the medium substantially in the middle of the cross-sectional area. This can be achieved by a measuring head being rotatably arranged at least approximately in the middle of the cross-sectional area and designed in such a way that the direction of the emitted radiation is inclined by an angle with respect to an axis running substantially parallel to the direction of flow of the medium and the rotational position of the direction of the radiation around the axis is variable.

The introduction of the measuring apparatus, or at least parts thereof, into the measuring volume, advantageously into the middle of the measuring volume (for example into the middle of a pipe), has the effect that the measuring accuracy is advantageously increased. The measuring distance is reduced (in the case of the arrangement in the middle of a pipe, the measuring distance is halved), so that more efficient measurements can be carried out. The rotatable arrangement of the measuring head (antenna), the sensor or the sensors advantageously makes it possible to record inhomogeneities of the flowing medium. Depending on the angle of the radiation in relation to the direction of flow, it is possible in particular to detect inhomogeneities perpendicularly to the direction of flow. Furthermore, advantages can be obtained by the spatial assignment of inhomogeneities, for example, in the splitting of flows. The rotation of the measuring sensor has the effect that inhomogeneities are reliably recorded. In a way similar to radar, the entire measuring volume can be scanned by means of the radiation, in order in this way to determine properties of the medium, in particular properties of a mixture, such as for example mass flow or particle burden. In the case of coal dust streamers, the spatial position of the coal dust streamers can be recorded exactly by means of a measuring arrangement in the middle of a pipe. A great advantage of this arrangement is that, instead of multiple sensors attached around the circumference of the pipe, only a single sensor has to be installed in the middle of the pipe, which means a reduction in the instrumentation, the installation costs and the maintenance costs.

The spatial measuring resolution is further improved if the emitted radiation is correspondingly shaped. In particular, a fan shape or a cone shape of the beam has proven to be particularly advantageous. In this particularly advantageous variant of an embodiment of the apparatus, means for shaping the emitted radiation are therefore present.

If multiple measuring signals are required to determine a property of the medium, in particular in the case of velocity measurements either of individual particles or of flows, the evaluation device is designed for processing measuring signals that are generated at different angles of inclination of the direction of radiation. In this advantageous variant of an embodiment, either multiple sensors are used or one sensor, which is designed for picking up multiple measuring signals.

In further advantageous exemplary embodiments, the rotation of the sensor takes place either continuously or incrementally. The setting of the rotational velocity and the rotational range will generally depend on the type of medium to be investigated, and in particular on the rate of change of the spatial distribution of the medium to be investigated.

In a further exemplary embodiment, the sensor comprising the transmitting device and the receiving device or the measuring head, which only comprises the device for coupling the radiation in and out, is movably arranged. This means a movement of the device both in the direction of flow and transversely thereto. Problem areas within the measuring volume can consequently be monitored better. Eccentric positioning of the sensor or the measuring head in the cross section of a pipe of a coal dust line would likewise bring about improved measuring accuracy, for example when observing a coal streamer.

In further advantageous variants of an embodiment, the sensor comprising the transmitting device and the receiving device is combined with a drive unit, which is intended to set the sensor in rotation, to form a media-tightly encapsulated module. All of the variants of an embodiment concerning the module have the advantage that the sensor or sensors is or are protected from influences of the medium, and consequently the risk of wear is reduced.

In further advantageous variants of an embodiment, means for conditioning the flow medium are present. These may be flaps, buoy-like inserts or other devices which, though independent of the measuring sensor, can be used on the basis of the measuring results to influence the flow conditions. In this way, measures for optimizing the flow can be taken, which is ultimately likewise conducive to improving the measuring accuracy.

### BRIEF DESCRIPTION

Some of the embodiments will be described in detail, with reference to the following figures, wherein like designations denote like members, wherein:

FIG. 1A shows a schematic representation of a measuring arrangement for determining properties of a flowing two-phase mixture from the prior art;

FIG. 1B shows a schematic representation of a number of cross sections from FIG. 1A;

FIG. 2A shows a first schematic representation of an embodiment of a measuring arrangement;

FIG. 2B shows a schematic representation of a cross section from FIG. 2A;

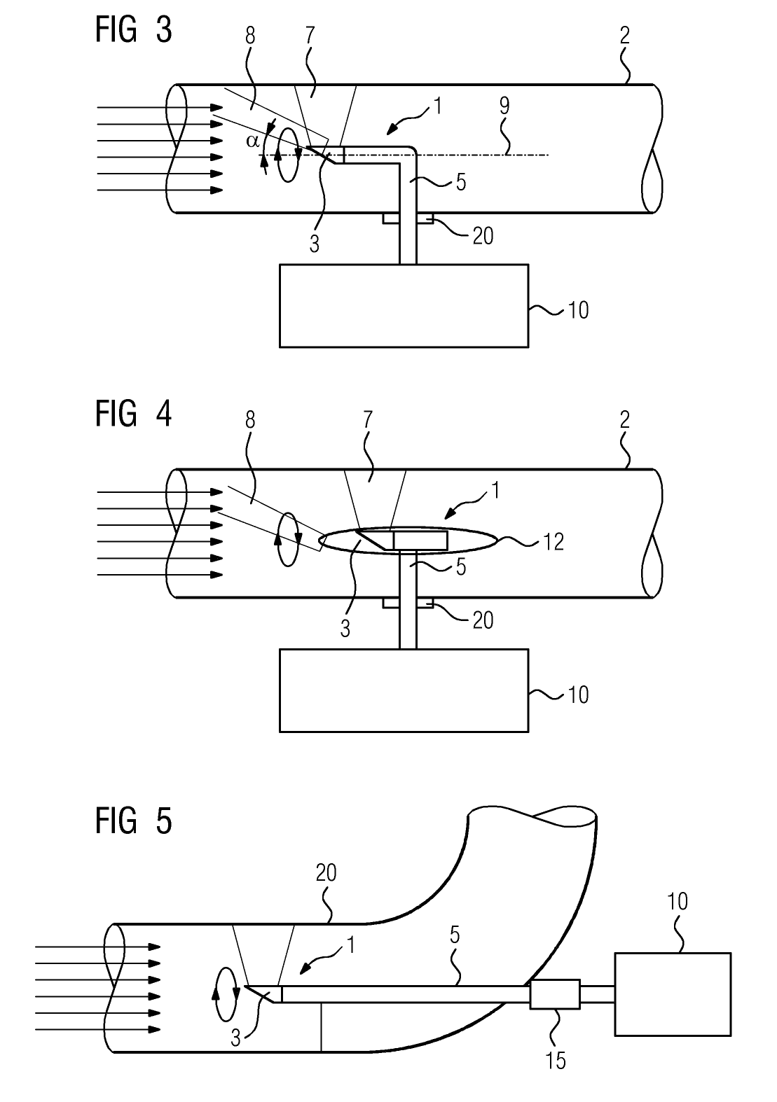

FIG. 3 shows a second schematic representation of an embodiment of the apparatus;

FIG. 4 shows a schematic representation of an embodiment of the apparatus; and

FIG. 5 shows a schematic representation of an embodiment of the apparatus when arranged in the vicinity of a pipe bend.

### DETAILED DESCRIPTION

FIGS. 1A and 1B shows a schematic representation of a microwave measuring arrangement for determining the burden of a two-phase flow from the prior art.

FIGS. 2A and 2B schematically shows a first exemplary embodiment of the apparatus according to the invention. In FIG. 2A, a section 2 of a straight pipeline with a circular cross section is represented in side view. In FIG. 2B, the section of pipe is represented at the point X in cross section.

Physically, the section of pipe 2 can be regarded as a hollow conductor, through which for example a multi-phase mixture of a gaseous carrier medium and extremely small solid particles flows. The flow is indicated by arrows at the left edge of the figure.

The measuring apparatus 1 according to embodiments of the invention comprises at least one measuring head 3, which is rotatably arranged approximately in the middle of the cross-sectional area of the hollow conductor. Here, the measuring head is a device for coupling in and out, for example an antenna. In this exemplary embodiment, the signal received is conducted by way of a signaling tube 5 that is bent by 90° in the direction of flow and is coupled out by way of the rotatable measuring head 3 transversely or at an angle a to the direction of flow. A turning device (not represented any more specifically here) and cable connections may be accommodated inside the signaling tube 5. In this exemplary embodiment, the signaling tube 5 also comprises a waveguide, for example a hollow conductor, for the signal received. The signaling tube 5 establishes the connection to a unit 10, which here comprises a means for generating and detecting the radiation used, the electronic signal processing and possibly also means for cooling or ventilation. These may alternatively also be arranged in the vicinity of the turning device with the measuring head 3 or in the measuring head 3. In this exemplary embodiment, a media-tight seal 20 is also arranged at the point where the signaling tube 5 enters the pipe.

In a way that is similar in principle to the principle of a radar device, the measuring head 3 emits shaped radiation 7 as a primary signal and receives the echo reflected within the flowing medium as a secondary signal. It corresponds to the radiation reflected at various surfaces, and as a result changed in its frequency, amplitude and/or phase position. In a specific case, the radiation transmitted can also be detected. The radiation received is subsequently converted into an electrical signal and passed on to a signal evaluation device (unit 10) and evaluated on the basis of various criteria. In this way, items of information concerning the medium to be investigated can be obtained. The measuring signal is always taken here as referring to the electronically converted secondary signal.

The sensor comprises at least one transmitting device and at least one receiving device for electromagnetic and/or acoustic radiation. The type of radiation is dependent here on the application. For applications in a coal dust line, microwave radiation is preferably used. For other applications, devices for emitting and detecting radiation in the visible wavelength range of the electromagnetic spectrum are conceivable, or devices for generating, coupling in and receiving ultrasound. The exact design and arrangement of the sensor (in the direct vicinity of the measuring head 3 or in the unit 10) are likewise dependent on the application. Transmitting and receiving devices may be combined to form a module or be implemented individually. Case-dependently, multiple transmitting and receiving devices may also be combined. In principle, the transmitting and receiving device comprises all of the means for generating radiation (such as laser diodes or microwave transmitters), means for coupling in and out (lenses), for example into a waveguide, waveguides and means for detecting the radiation (such as a photodetector or microwave receiver). The measuring head is always understood here as meaning only the means for coupling radiation into and out of the medium.

In the variant of an embodiment outlined in FIGS. 2A and 2B, the radiation 7 emitted from the transmitting device has a spatial extent, meaning that the beam does not emerge linearly from the radiation source but is widened by a means for shaping or influencing the radiating characteristics, such as for example a diffusing lens, or a horn radiator. Apart from shaping into a divergent beam, shaping into a parallel bundle of rays or focusing may also be advantageous. Spatial widening of the radiation transmitted has the effect of improving the spatial resolution of the measuring arrangement, in particular transversely to the direction of flow of the medium, because a greater cross section is covered.

This is clear in particular from FIG. 2B. In FIG. 2B, a cross-sectional view of the point X of the section of pipe is schematically represented. A sensor arrangement held by means of the stand 5 is not represented. The rotation of the sensor or the measuring head is indicated by the circular arrow. The angle cp is taken as referring to the rotational angle or rotational position. In a way similar to radar, a fanned-out beam 7 emitted from the transmitting device passes over the measuring cross section. The cross section of the streamer designated by SQ, for example of a coal dust streamer, lies at least partially within the shaded measuring cone 7 for a period of time, so that throughout this period of time a signal can be detected and can also be spatially assigned.

According to embodiments of the invention, the measuring head 3 is arranged rotatably about an axis running substantially parallel to the direction of flow of the multi-phase mixture. In FIG. 2A, this is the axis of symmetry 9 in the longitudinal direction of the pipeline, which intersects the cross section of the pipe approximately at its middle point. Depending on the application case, some other longitudinal axis may also be advantageous, so that the sensor is arranged eccentrically in the cross-sectional view. The more movable the sensor is in the longitudinal and transverse directions, the better inhomogeneities within the measuring volume can be recorded.

The rotation of the measuring head may be achieved for example by a small motor, which drives a shaft on which in turn the sensor is attached; it is also possible moreover for a compressed-air or electromechanical drive to be provided. Furthermore, a position sensor for the rotational position should be provided. The turning device is preferably arranged inside the stand or signaling tube 5. If the sensor itself rotates, the direction of rotation must be regularly reversed in order to prevent lines from becoming twisted.

In FIG. 2A, the angle between the emitted radiation and the direction of flow of the medium is approximately 90°. This angle has proven to be advantageous, in particular with respect to the investigation of coal dust streamers. In the most general case, however, the measuring head or sensor 3 is designed as in FIG. 3 in such a way that the direction of the radiation 8 emitted from the transmitting device is inclined by an angle a with respect to the axis 9 running parallel to the direction of flow of the medium. This can be achieved for example by a pivoting head.

If the sensor is designed in such a way that, after the emission of two primary signals 7 and 8, two measuring signals are picked up, with a known sensor position or measuring head position it is also possible for velocity measurements, for example of small solid particles, to be carried out. The relative movement between the transmitter and the object can similarly be used to determine the particle velocity from the frequency shift of the reflected signal by the Doppler effect. The successive performance of individual measurements produces the distance covered and the absolute velocity of an object. Furthermore, with a known sensor position, angles, directions and distances from certain objects, such as larger solid particles, are possible.

The evaluation device 10 determines a property or a number of properties of the medium on the basis of the measuring signal received. In an exemplary embodiment, for example, the proportion of the solid matter in a dust mixture is determined for a two-phase mixture by means of microwave radiation. Simultaneous recording of the rotational position likewise takes place for the angle-dependent representation of the measuring results. Depending on the requirement, measurements may be taken continuously or at time intervals. A representative overall result is obtained by means of averaging over time, depending on how high the rotational velocity of the sensor is.

The evaluation device 10 may be connected to an open-loop or closed-loop control for optimizing the process. A combination with an intelligent and/or self-adjusting final controlling element may for example be integrated in a control system for controlling an automation process.

Furthermore, means for conditioning the flow may be used either inside the flow channel or as part of the apparatus 1 according to embodiments of the invention.

In a further exemplary embodiment according to FIG. 4, the sensor is in a media-tight and securely mounted, encapsulated module 12 within the measuring volume, or here the pipeline 2, whereby the mechanical measuring setup is simplified, though depending on operating conditions an external energy supply must be provided. The sensor 3 comprises the transmitting device and the receiving device; in addition, there is a drive unit, which sets the sensor in rotation. In the exemplary embodiment shown, this module 12 is of a streamlined design, in order to achieve a measuring result that is as undisturbed as possible. Inside the encapsulated module, means for cooling and/or ventilation may be additionally provided. These may alternatively also be realized outside the module, and coolant or air may be fed in by a hose connection. The protective casing of such a module 12 should be of a wear-resistant and media-tight design. In particular, it must be transmissive to the radiation that is respectively used. Furthermore, as in the other variants of an embodiment, further means may be present inside or outside the module, for example means for reporting a rotational position, control flaps for conditioning the flow or the radiation used. If a second sensor is provided, for example for determining the velocity, it too is arranged with all necessary additional devices inside the module. Particularly advantageous in the case of this variant of an embodiment is the improved possibility of fastening the module 12 to a straight stand or signaling tube 5.

In FIG. 5, a section 20 of a bent pipeline with a circular cross section, in which the apparatus 1 according to embodiments of the invention is arranged, is represented in side view. In this variant of an embodiment, the sensor 3 is for example arranged at one end of a predominantly straight stand or signaling tube 5. In this exemplary embodiment, the signaling tube 5 is introduced through a bore in the pipe bend into the interior of the pipe and connects the sensor 3 to the evaluation unit 10, which is arranged outside the pipe. The advantage of this variant is that it can be mounted particularly easily and can be displaced and turned manually or mechanically from the outside. This setup can be installed particularly easily. Depending on the length of the signaling tube 5, a supporting structure is necessary for securement. Furthermore, it must be ensured that the signaling tube is connected by way of a media-tight closure 15 or a media-tight seal, such as for example a flange.

Although the present invention has been disclosed in the form of preferred embodiments and variations thereon, it will be understood that numerous additional modifications and variations could be made thereto without departing from the scope of the invention.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

1. An apparatus for determining properties of a dust mixture flowing through a cross-sectional area of a coal dust line, comprising:
at least one sensor, which has at least one transmitting device for coupling electromagnetic and/or acoustic radiation into the dust mixture and at least one receiving device for generating a measuring signal on a basis of at least one of radiation reflected in the dust mixture and radiation transmitted by the dust mixture;
an evaluation device, which determines a property in the dust mixture on a basis of the measuring signal, wherein the at least one transmitting device and the at least one receiving device or a measuring head are rotatably arranged at least approximately in a middle of the cross-sectional area and are designed in such a way that a direction of the emitted radiation is inclined by an angle with respect to an axis running substantially parallel to a direction of flow of the dust mixture and a rotational position of the direction of the radiation around the axis is variable.

2. The apparatus as claimed in claim 1, wherein means for shaping the emitted radiation are also present.

3. The apparatus as claimed in claim 1, wherein the evaluation device is designed for determining a property of the dust mixture on the basis of measuring signals that are generated at different angles of inclination of the direction of radiation, it being possible in this way for velocity measurements particular to be carried out.

4. The apparatus as claimed in claim 1, wherein the rotational position is changed at least one of continuously and incrementally.

5. The apparatus as claimed in claim 1, wherein the at least one sensor comprising the at least one transmitting device and the at least one receiving device or the measuring head is movably arranged.

6. The apparatus as claimed in claim 1, wherein the at least one sensor comprising the at least one transmitting device and the at least one receiving device is combined with a drive unit, which is intended to set the at least one sensor in rotation, to form a media-tightly encapsulated module.

7. The apparatus as claimed in claim 1, wherein, inside an encapsulated module, means for cooling and/or ventilation are additionally provided.

8. The apparatus as claimed in claim 1, wherein the angle of the emitted radiation with respect to the direction of flow of the dust mixture is approximately 90°.

9. The apparatus as claimed in claim 1, wherein the evaluation device is connected to an open-loop and/or closed-loop control.

10. The apparatus as claimed in claim 1, wherein connections to further means for conditioning the flow of the dust mixture are provided.

11. The apparatus as claimed in claim 1, wherein the radiation to be used lies in a microwave range, an optical wavelength range or an acoustic wavelength range.

12. (canceled)
